(19) United States
(12) Patent Application Publication
(10) Pub. No.: US 2015/0196676 A1
(43) Pub. Date: Jul. 16, 2015

(54) SYSTEM FOR REMOTELY CONTROLLING AND MANAGING STERILIZER

(71) Applicant: HANSHIN MEDICAL Co., Ltd., Incheon (KR)

(72) Inventor: Jung Yeul KIM, Incheon (KR)

(73) Assignee: HANSHIN MEDICAL Co., Ltd., Incheon (KR)

(21) Appl. No.: 14/595,556

(22) Filed: Jan. 13, 2015

(30) Foreign Application Priority Data
Jan. 13, 2014 (KR) 10-2014-0004004

(51) Int. Cl. A61L 2/24; A61L 2/16

(52) U.S. Cl. CPC A61L 2/24; A61L 2/16

(57) ABSTRACT

Disclosed herein is a system for remotely controlling and managing a sterilizer. The system for remotely controlling and managing a sterilizer according to an embodiment of the present invention includes N sterilizers configured to sterilize sterilization targets, a mobile terminal configured to receive a control command for each sterilizer or display information related to the sterilizer, and an integrated sterilizer management server connected to the N sterilizers over a network, and configured to transmit the control command received from the mobile terminal to the corresponding sterilizer, allow the corresponding sterilizer to be driven in compliance with the control command, collect current status information of the N sterilizers, and transmit the collected current status information to the mobile terminal.

### BACKGROUND OF THE INVENTION

1. Field of the Invention

The present invention relates to a system for remotely controlling and managing a sterilizer, which can integrally control various sterilizers that are operated so as to sterilize various types of devices or articles while remotely controlling the operations of sterilizers from a remote place where the sterilizers are not located, by using a user's mobile terminal capable of accessing a management server.

2. Description of the Related Art

Generally, sterilizers have been used to sterilize medical appliances and medical supplies used to medically treat patients, especially, medical instruments such as a surgical knife and operating scissors.

Typical sterilizers are problematic in that a user must control the operation of each sterilizer by personally manipulating a control button or the like installed on the sterilizer, as disclosed in Korean Patent Application Publication No. 10-2011-0043933 (Date of publication: Apr. 28, 2011).

### SUMMARY OF THE INVENTION

Accordingly, the present invention has been made keeping in mind the above problems occurring in the prior art, and an object of the present invention is to provide a system for remotely controlling and managing a sterilizer, which is configured, by utilizing a mobile terminal from a place where the sterilizer is not located before a daily task is started, such that a required operation before sterilization, such as preheating, is performed by turning on the operating power of the sterilizer, or such that, if necessary, the status of the sterilizer can be determined in advance by activating a test cycle, such as a vacuum leakage cycle enabling the presence or absence of an abnormality in equipment to be checked.

Another object of the present invention is to provide a system for remotely controlling and managing a sterilizer, which is configured, by utilizing a mobile terminal after a sterilization target has been inserted and a sterilization cycle has been activated at a time point at which a daily task is terminated, such that the status of the sterilization cycle can be remotely and externally monitored, and such that if it is determined that the sterilization cycle has been completed, the sterilizer is controlled so that the operating power of the sterilizer is remotely turned off, thus maximizing the usage convenience and operation efficiency of the system.

Other objects and advantages of the present invention will be understood from the following descriptions, and will be more clearly learned from embodiments of the present invention. Further, it may be easily understood that the objects and advantages of the present invention may be practiced by means described in the accompanying claims and combinations thereof.

In accordance with an embodiment of the present invention, there is provided a system for remotely controlling and managing sterilizer, including N sterilizers configured to sterilize sterilization targets, a mobile terminal configured to receive a control command for each sterilizer or display information related to the sterilizer, and an integrated sterilizer management server connected to the N sterilizers over a network, and configured to transmit the control command received from the mobile terminal to the corresponding sterilizer, allow the corresponding sterilizer to be driven in compliance with the control command, collect current status information of the N sterilizers, and transmit the collected current status information to the mobile terminal.

The integrated sterilizer management server may include a management server for processing and managing commands related to the sterilizers, and middleware for relaying information stored in or managed by the management server.

The management server may include a sterilizer communication data transmission/reception module for transmitting/receiving data to/from the N sterilizers in real time, a sterilizer type classification module for analyzing data received from the N sterilizers, classifying types of the sterilizers and registration or non-registration of the sterilizers, and converting data to be transmitted to a corresponding sterilizer into a data format corresponding to a type of sterilizer if there is data to be transmitted to the sterilizer, a monitoring data processing module for analyzing the data received from the N sterilizers, acquiring information about at least one of processing status, Input/Output (I/O) status, power status, alarm status, and cycle information of the corresponding sterilizer, and storing the acquired information in a given database (DB), a control command data processing module for classifying control commands for the sterilizers, determining whether the sterilizers are controllable, processing transmitted/received control commands, and storing results of control based on the transmitted/received control commands in the DB, a user interface module for setting sterilizers or users, and displaying cycles, alarm or control status, a sterilization cycle management module for, when a sterilization cycle-related request command is input, searching the DB for respective periods/sterilizer types/sterilizer numbers in relation to a sterilization cycle, and extracting found data based on the search, an alarm management module for, when a current sterilizer alarm condition checking command is input, searching for and extracting current alarm occurrence conditions of the sterilizers for respective periods/sterilizer types/sterilizer numbers, a setting management module for managing registration/change/deletion of sterilizers, registration/change/deletion of users, or configuration of the management server, a control management module for, when a sterilizer control history search command is input, searching for and extracting control history and control results for respective periods/sterilizer types/sterilizer numbers, and the DB for storing data used by the remote sterilizer control and management system.

The middleware may include a DB interface module used to access the DB of the management server, a power control module configured to transmit a command for power control from a user to the management server or receive results of power control from the management server, a cycle control module configured to transmit a command for cycle control from the user to the management server or receive results of cycle control from the management server, a monitoring module configured to receive real-time operating status of a sterilizer selected by the user from the management server, an alarm processing module configured to transmit alarm occurrence information to a registered mobile terminal when an alarm occurs in a specific sterilizer, a user authentication module configured to process user authentication, and a communication data processing module configured to be capable of transmitting/receiving data to/from the mobile terminal in real time and convert data to be transmitted/received in conformity with a data format of the mobile terminal.

Each of the sterilizers may include a main control module including a second transmission/reception unit capable of transmitting/receiving data to/from the integrated sterilizer management server when operating power of the main control module is turned on, a monitoring device for monitoring status of the sterilizer, a main control unit for controlling an operation of the sterilizer, and a power status output unit for outputting a power control command for the main control module, and a power control module including a first transmission/reception unit capable of transmitting/receiving data to/from the integrated sterilizer management server when the operating power of the main control module is turned off, a power supply switching unit for supplying power to the main control module or cutting off the supply of power to the main control module in compliance with a control command from a power control unit, a power status monitoring unit for monitoring a power control command for the main control module, output from the power status output unit, and the power control unit for controlling the power supply switching unit so that power is supplied to the main control module or the supply of the power to the main control module is cut off in compliance with the power control command for the main control module, received from the integrated sterilizer management server or the power status monitoring unit.

The mobile terminal may include a user input unit capable of receiving a command required to control an operation of a corresponding sterilizer.

The mobile terminal may be configured to, when a test cycle command for activating a cycle enabling status of each sterilizer to be inspected is remotely input before a sterilization cycle is activated, transmit the input test cycle command to the integrated sterilizer management server, receive status information of the sterilizer from the integrated sterilizer management server, and display the status information of the sterilizer.

### BRIEF DESCRIPTION OF THE DRAWINGS

FIG. 1 is a diagram showing a system for controlling and managing a sterilizer according to an embodiment of the present invention;

FIG. 2 is a diagram showing in detail an integrated sterilizer management server according to an embodiment of the present invention;

FIG. 3 is a diagram showing in detail a sterilizer according to an embodiment of the present invention; and

FIG. 4 is a diagram showing the setting of a scheduling cycle according to an embodiment of the present invention.

### DESCRIPTION OF THE PREFERRED EMBODIMENTS

Hereinafter, embodiments of the present invention will be described in detail with reference to the attached drawings.

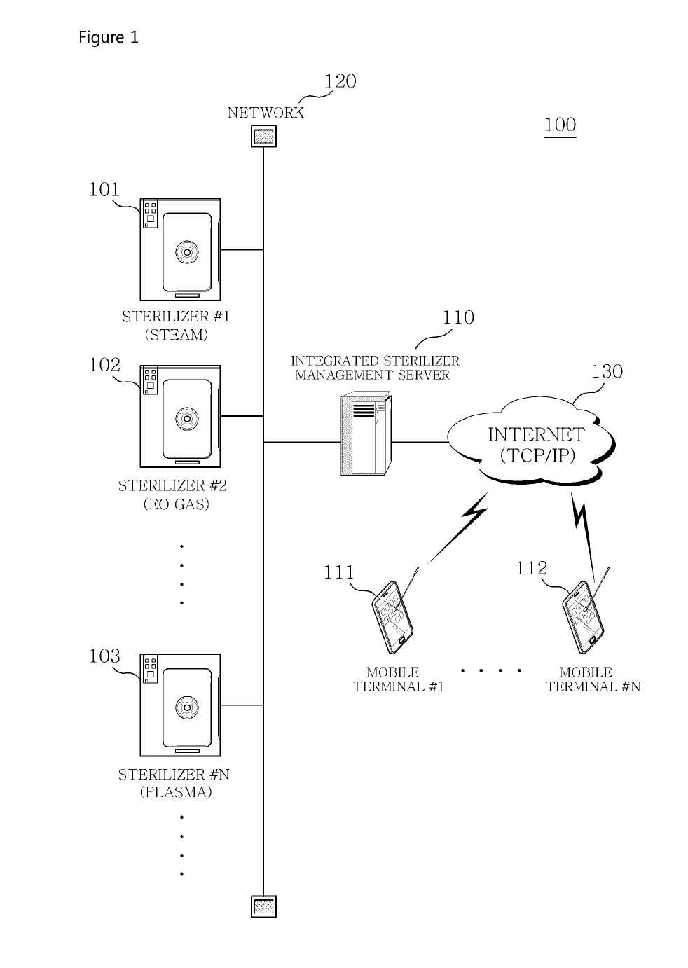

FIG. 1 is a diagram showing a system for remotely controlling and managing a sterilizer (hereinafter referred to as a “remote sterilizer control and management system”) according to an embodiment of the present invention.

Referring to FIG. 1, a remote sterilizer control and management system 100 includes N sterilizers 101, 102, and 103, an integrated sterilizer management server 110, and mobile terminals 111 and 112.

The sterilizers 101, 102, and 103 may sterilize sterilization targets. The sterilizers 101, 102, and 103 may be of various types, such as a steam sterilizer, an Ethylene Oxide (EO) gas sterilizer, a plasma sterilizer, and a dry sterilizer, depending on the types of sterilization targets so as to sterilize medical appliances or supplies used in hospitals.

The integrated sterilizer management server 110 is connected to the N sterilizers 101, 102, and 103 over a network 120, and is capable of transmitting/receiving various types of data to/from the sterilizers 101, 102, and 103.

The integrated sterilizer management server 110 transmits a control command input from the mobile terminal 111 or 112 to the corresponding sterilizer 101, 102, or 103. The corresponding sterilizer 101, 102, or 103 may be driven in compliance with a control command received from the integrated sterilizer management server 110. When the control command is, for example, a command for turning on the power of the corresponding sterilizer, power is applied to the corresponding sterilizer 101, 102, or 103 to turn on the sterilizer. In this way, by using the mobile terminal 111 or 112, the operation of each sterilizer 101, 102, or 103 may be remotely controlled.

The integrated sterilizer management server 110 may collect information about the current status of the N sterilizers 101, 102, and 103 and may transmit the collected information to the mobile terminal 111 or 112.

The mobile terminal 111 or 112 may receive control commands for the sterilizers 101, 102, and 103, or display information related to the sterilizers 101, 102, and 103.

The control commands may include commands for power ON/power OFF/power ON and OFF scheduling, commands for sterilization cycle start/test cycle start/cycle stop, commands for scheduled cycle setting/scheduled cycle cancellation, etc.

The mobile terminal 111 or 112 is configured to, when a test cycle command for activating a cycle in which the status of the corresponding sterilizer can be inspected before a sterilization cycle is activated is input from a remote place, transmit the input test cycle command to the integrated sterilizer management server 110, receive the status information of the sterilizer from the integrated sterilizer management server 110, and display the status information. In this case, the integrated sterilizer management server 110 may transmit the test cycle command to the corresponding sterilizer. The corresponding sterilizer may transmit the status information of the sterilizer to the integrated sterilizer management server 110 either while executing the test cycle, or after completing the execution of the entire test cycle. The integrated sterilizer management server 110 may transmit the status information of the sterilizer to the mobile terminal 111 or 112.

For example, each mobile terminal 111 or 112 may include any of a mobile phone, a smart phone, a Personal Digital Assistants (PDA) terminal, and a Portable Multimedia Player (PMP), and may include, as components, a user input unit for generating input data required to control the operation of the terminal, or receiving a command required to control the operation of the sterilizer and a display unit for displaying information.

An example of the driving process of the remote sterilizer control and management system 100 will be described below. A user may execute a sterilizer control program installed on the mobile terminal 111 and may access the integrated sterilizer management server 110. The user may complete user authentication using the mobile terminal 111, and then select a sterilizer (e.g., 102) desired to be remotely controlled from among the N sterilizers 101, 102, and 103 that are connected to the integrated sterilizer management server 110. The user inputs a sterilizer control command using the mobile terminal 111, and the mobile terminal 111 may transmit the input sterilizer control command to the integrated sterilizer management server 110. The integrated sterilizer management server 110 may transmit the received sterilizer control command to the selected sterilizer 102. The sterilizer 102 may be operated in compliance with the received sterilizer control command. The sterilizer 102 transmits the results of control to the integrated sterilizer management server 110. The integrated sterilizer management server 110 may store the received control results in a database (DB) and transmit the received control results to the mobile terminal 111.

By utilizing the mobile terminal from a place where the sterilizer is not located before a daily task is started, the remote sterilizer control and management system is configured such that a required operation before sterilization, such as preheating, is performed by turning on the operating power of the sterilizer, or such that, if necessary, the status of the sterilizer may be determined in advance by activating a test cycle, such as a vacuum leakage cycle enabling the presence or absence of an abnormality in equipment to be checked.

Further, by utilizing the mobile terminal after a sterilization target has been inserted and a sterilization cycle has been activated at a time point at which a daily task is terminated, the remote sterilizer control and management system is configured such that the status of the sterilization cycle may be remotely and externally monitored, and such that if it is determined that the sterilization cycle has been completed, the sterilizer is controlled so that the operating power of the sterilizer is remotely turned off, thus maximizing the usage convenience and operation efficiency of the system.

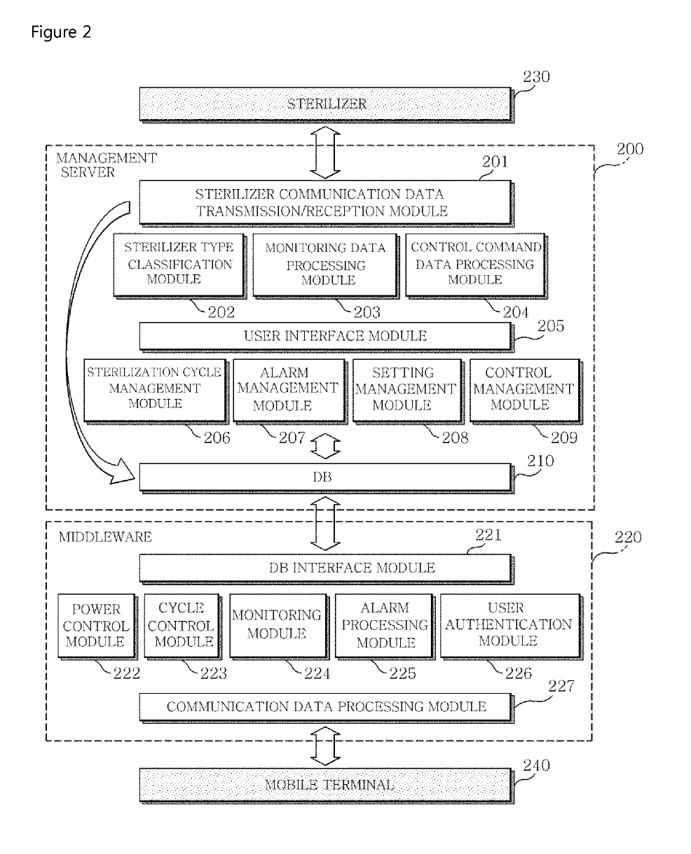

FIG. 2 is a diagram showing in detail the integrated sterilizer management server according to an embodiment of the present invention.

Referring to FIG. 2, the remote sterilizer control and management system includes an integrated sterilizer management server, a sterilizer 230, and a mobile terminal 240.

The integrated sterilizer management server may include a management server 200 and middleware 220.

The management server 200 may process and manage commands related to sterilizers.

The management server 200 includes a sterilizer communication data transmission/reception module 201, a sterilizer type classification module 202, a monitoring data processing module 203, a control command data processing module 204, a user interface module 205, a sterilization cycle management module 206, an alarm management module 207, a setting management module 208, a control management module 209, and a DB 210.

The sterilizer communication data transmission/reception module 201 may transmit/receive data to/from N sterilizers in real time.

The sterilizer type classification module 202 may analyze data received from the N sterilizers and then classify the types of sterilizers and whether sterilizers have been registered. When data to be transmitted to the sterilizer is present, the sterilizer type classification module 202 may convert the data to be transmitted into a data format corresponding to the type of sterilizer.

The monitoring data processing module 203 may analyze the data received from the N sterilizers, acquire at least one of the processing condition, Input/Output (I/O) status, power status, alarm status, and cycle information of the corresponding sterilizer, and store the acquired information in a given DB.

The control command data processing module 204 may classify control commands for sterilizers, determine whether the sterilizers are controllable, and manage control results based on transmitted/received control commands, and may also store the control results based on the transmitted/received control commands in the DB.

The user interface module 205 may allow sterilizers or users to be registered/deleted/changed with respect to the management server 200 or display check cycle, alarm, or control status so that a worker may check the cycle, alarm, or control status.

When a request command related to a sterilization cycle is input, the sterilization cycle management module 206 may search the DB for respective periods/sterilizer types/sterilizer numbers in relation to the sterilization cycle, and may extract the corresponding data. For example, when a command for requesting the viewing of information related to the sterilization cycle is input, the sterilization cycle management module 206 may search the DB for the corresponding information and extract the found information. The extracted information is transmitted to the mobile terminal 240. The mobile terminal 240 displays the extracted information and then the user may check the displayed information.

If a checking command for a current sterilizer alarm condition is input, the alarm management module 207 may search for and extract current alarm occurrence conditions for respective periods/sterilizer types/sterilizer numbers. For example, when a command required to check the current conditions of sterilizer alarms that have occurred to date is input, the alarm management module 207 may search for and extract the current conditions of occurrence of sterilizer alarms for respective periods/sterilizer types/sterilizer numbers. The extracted information is transmitted to the mobile terminal 240. The mobile terminal 240 displays the received extracted information, and the user may check the displayed information.

The setting management module 208 may manage the registration/change/deletion of sterilizers, the registration/change/deletion of users, or the configuration of the management server. For example, when sterilizer or user setting information is input, the setting management module 208 may systematically store and manage the setting information in the DB.

When a search command for sterilizer control history is input, the control management module 209 may search for and extract control history and control results for respective periods/sterilizer types/sterilizer numbers. For example, when a command to search for the control history of sterilizers stored to date is input, the control management module 209 may search for and extract control history and control results for respective periods/sterilizer types/sterilizer numbers. The extracted information is transmitted to the mobile terminal 240. The mobile terminal 240 displays the extracted information, and the user may check the displayed information.

The DB 210 may store all types of data used in the remote sterilizer control and management system according to the present invention.

The middleware 220 may relay information stored or managed in the management server.

The middleware 220 includes a DB interface module 221, a power control module 222, a cycle control module 223, a monitoring module 224, an alarm processing module 225, a user authentication module 226, and a communication data processing module 227.

The DB interface module 221 is used to access the DB 210 of the management server. For example, when a command to access the information stored in the DB 210 of the management server 200 is received from the mobile terminal 240 through the communication data processing module 227, the middleware 220 may access the corresponding information through the DB interface module 221.

The power control module 222 may transmit a command for power control from the user to the management server 200 or receive the results of power control from the management server 200. The management server 200 may transmit the command for power control, received from the power control module 222, to the sterilizer 230 through the communication data transmission/reception module 201. The middleware 220 transmit the results of power control, received from the power control module 222, to the mobile terminal 240 through the communication data processing module 227.

The cycle control module 223 may transmit a command for cycle control from the user to the management server 200 or receive the results of cycle control from the management server 200. The term “cycle control” denotes the control of a cycle activated by each sterilizer.

The monitoring module 224 may receive the real-time operating status of a sterilizer selected by the user from the management server 200. The received information may be transmitted to the mobile terminal 240 through the communication data processing module 227.

When an alarm occurs in a specific sterilizer, the alarm processing module 225 may transmit alarm occurrence information to a registered mobile terminal. For example, when an alarm occurs in a third sterilizer, if the alarm processing module 225 transmits alarm occurrence information, indicating that the alarm has occurred in the corresponding sterilizer, to the communication data processing module 227, the communication data processing module 227 may transmit the received alarm occurrence information to the registered mobile terminal.

The user authentication module 226 may process user authentication. For example, the user authentication module 226 may authenticate the ID and password of the user and manage the log-in/out activity of the user when the user desires to access the integrated sterilizer management server using his or her mobile terminal.

The communication data processing module 227 may transmit/receive data to/from the mobile terminal 240 in real time, and may convert the data to be transmitted/received in conformity with the data format of the mobile terminal.

By utilizing the mobile terminal from a place where the sterilizer is not located before a daily task is started, the remote sterilizer control and management system is configured such that a required operation before sterilization, such as preheating, is performed by turning on the operating power of the sterilizer, or such that, if necessary, the status of the sterilizer may be determined in advance by activating a test cycle, such as a vacuum leakage cycle enabling the presence or absence of an abnormality in equipment to be checked.

Further, by utilizing the mobile terminal after a sterilization target has been inserted and a sterilization cycle has been activated at a time point at which a daily task is terminated, the remote sterilizer control and management system is configured such that the status of the sterilization cycle may be remotely and externally monitored, and such that if it is determined that the sterilization cycle has been completed, the sterilizer is controlled so that the operating power of the sterilizer is remotely turned off, thus maximizing the usage convenience and operation efficiency of the system.

Furthermore, if a scheduling cycle enabling a cycle to be activated at a time set by the user is used, the remote sterilizer control and management system may take out and use a sterilization target at a designated time, so that there is no need to necessarily operate the sterilizer in the presence of the sterilizer at the time at which a sterilization cycle is to be started so as to keep the time of use, thus improving the convenience of use.

Furthermore, the remote sterilizer control and management system allows a plurality of managers to access the management server through their mobile terminals at a place where sterilizers are not located and to check the processing status of a currently activated sterilization cycle in real time.

Furthermore, when an abnormality occurs during the performance of a specific procedure/step operation of a sterilization cycle in the absence of the user or managers, the remote sterilizer control and management system is configured such that the management server may immediately transmit the procedure in which the abnormality has occurred and details of the abnormality to mobile communication terminals of a plurality of persons in charge, thus promptly coping with the abnormality.

Furthermore, the remote sterilizer control and management system transmits an alarm to the mobile communication terminal of a person in charge either when sterilant (steam sterilizer: water, plasma sterilizer: peroxide, and EO gas sterilizer: EO gas) used for the sterilizer is exhausted or when the sterilant is determined to be insufficient before the sterilant is exhausted, thus allowing the user to previously acquire a sufficient amount of sterilant.

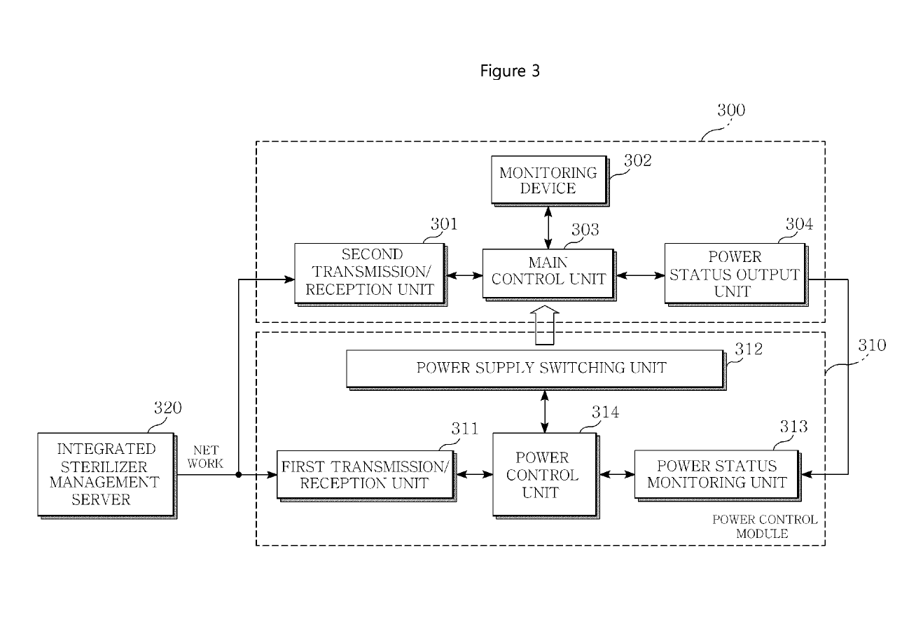

FIG. 3 is a diagram showing in greater detail a sterilizer according to an embodiment of the present invention.

Referring to FIG. 3, the sterilizer may include a main control module 300 and a power control module 310.

The main control module 300 includes a second transmission/reception unit 301, a monitoring device 302, a main control unit 303, and a power status output unit 304.

The second transmission/reception unit 301 may transmit/receive data to/from an integrated sterilizer management server 320 when the operating power of the main control module 300 is turned on. In contrast, when the operating power of the main control module 300 is turned off, the power of the second transmission/reception unit 301 is turned off, and thus it is impossible to transmit/receive data to/from the integrated sterilizer management server 320.

The monitoring device 302 may monitor the status of the sterilizer. For example, when the monitoring device 302 may include various types of sensors or sensing devices for detecting the status of the sterilizer or the peripheral devices thereof, such as a temperature sensor, a pressure sensor, a humidity sensor, and a water level sensor required for the operation of a sterilization cycle program.

The main control unit 303 may control the overall operation of the sterilizer. For example, the main control unit 303 may perform sterilization on a sterilization target using the user's settings or the settings of various types of sterilization cycle programs or the like basically stored in memory.

The main control unit 303 may transmit the operating status thereof to the management server in real time through the second transmission/reception unit 301, and may drive the sterilizer in compliance with a control command when the control command is received.

When a situation in which the operating power is to be turned off is reached, the main control unit 303 may perform control so that a power OFF switching command signal is output to the power status output unit 304.

The power status output unit 304 may output a power control command for the main control module 300. For example, the power status output unit 304 may output an operating power ON status maintenance command, an operating power OFF status maintenance command, an operating power OFF switching command (power ON->power OFF), etc. In other words, the power status output unit 304 may output commands for future power status of the main control module 300.

The power control module 310 includes a first transmission/reception unit 311, a power supply switching unit 312, a power status monitoring unit 313, and a power control unit 314.

The first transmission/reception unit 311 may transmit/receive data to/from the integrated sterilizer management server 320 when the operating power of the main control module 300 is turned off. In contrast, when the operating power of the main control module 300 is turned on, the power of the first transmission/reception unit 311 is turned off.

The power supply switching unit 312 may supply power to the main control module 300 or cut off the supply of power thereto in compliance with a control command from the power control unit.

The power status monitoring unit 313 may monitor a power control command for the main control module 300, output from the power status output unit 304.

The power control unit 314 may control the power supply switching unit 321 so that power is supplied to the main control unit 300 or the supply of the power is cut off in compliance with the power control command for the main control module 300, received from the integrated sterilizer management server 320 or the power status monitoring unit 313.

For example, when a command for turning on the operating power of the main control module 300 is input, the power control unit 314 may control the power supply switching unit 321 so that the operating power is supplied to the main control module 300. Further, the power control unit 314 may cut off the power of the first transmission/reception unit 311.

As another example, when a command for turning off the operating power of the main control module 300 is input, the main control unit 303 outputs the operating power OFF switching command through the power status output unit 304. When the operating power OFF switching command is received through the power status monitoring unit 313, the power control unit 314 may control the power supply switching unit 321 so that the supply of the operating power to the main control module 300 is cut off. Further, the power control unit 314 may supply power for the first transmission/reception unit 311. Accordingly, the power control unit 314 may receive a control command from the integrated sterilizer management server 320 through the first transmission/reception unit 311.

The sterilizer separately includes the main control module and the power control module, thus more efficiently controlling power. In addition, even when power is not applied to the main control module, the sterilizer may receive a control command using only a minimum of power without turning on the power of the main control module.

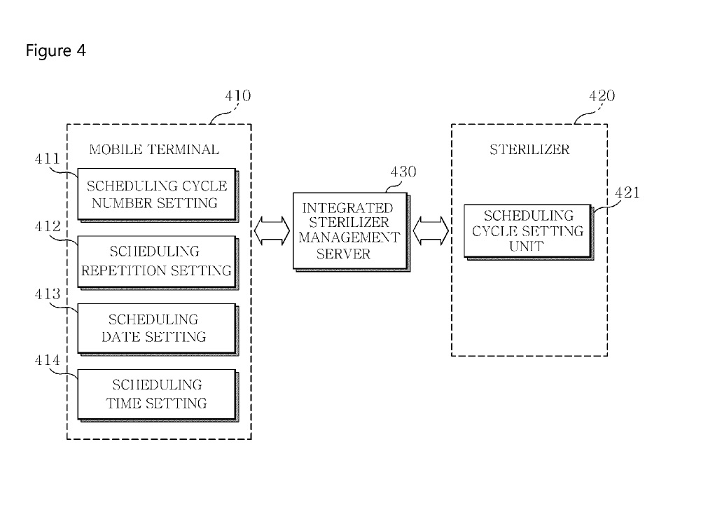

FIG. 4 a diagram showing the setting of a scheduling cycle according to an embodiment of the present invention.

Referring to FIG. 4, a user may perform various types of setting, such as the setting of a cycle program and the setting of a desired date repetition section, in addition to the setting 411 of a desired scheduling cycle number, the setting 412 of scheduling repetition, the setting 413 of scheduling date, and the setting 414 of scheduling time, via a scheduling cycle setting screen on a mobile terminal 400.

The user may personally set various scheduling cycles even through the scheduling cycle setting unit 421 of a sterilizer 420.

The integrated sterilizer management server 430 transmits a control command to the sterilizer 420 so that the sterilizer 420 may be driven depending on setting information input by the user through the mobile terminal 400 or the sterilizer 420. Accordingly, the sterilizer 420 may activate a scheduling cycle at a desired time.

In accordance with the disclosed invention, by utilizing a mobile terminal from a place where a sterilizer is not located before a daily task is started, the present invention is configured such that a required operation before sterilization, such as preheating, is performed by turning on the operating power of the sterilizer, or such that, if necessary, the status of the sterilizer may be determined in advance by activating a test cycle, such as a vacuum leakage cycle enabling the presence or absence of an abnormality in equipment to be checked.

Further, by utilizing a mobile terminal after a sterilization target has been inserted and a sterilization cycle has been activated at a time point at which a daily task is terminated, the present invention is configured such that the status of the sterilization cycle may be remotely and externally monitored, and such that if it is determined that the sterilization cycle has been completed, the sterilizer is controlled so that the operating power of the sterilizer is remotely turned off, thus maximizing the usage convenience and operation efficiency of the system.

Furthermore, if a scheduling cycle enabling a cycle to be activated at a time set by the user is used, the present invention may take out and use a sterilization target at a designated time, so that there is no need to necessarily operate the sterilizer in the presence of the sterilizer at the time at which a sterilization cycle is to be started so as to keep the time of use, thus improving the convenience of use.

Furthermore, the present invention allows a plurality of managers to access the management server through their mobile terminals at a place where sterilizers are not located and to check the processing status of a currently operated sterilization cycle in real time.

Furthermore, when an abnormality occurs during the performance of a specific procedure/step operation of a sterilization cycle in the absence of the user or managers, the present invention is configured such that the management server may immediately transmit the procedure in which the abnormality has occurred and details of the abnormality to mobile communication terminals of a plurality of persons in charge, thus promptly coping with the abnormality.

Furthermore, the present invention transmits an alarm to the mobile communication terminal of a person in charge either when sterilant (steam sterilizer: water, plasma sterilizer: peroxide, and EO gas sterilizer: EO gas) used for the sterilizer is exhausted or when the sterilant is determined to be insufficient before the sterilant is exhausted, thus allowing the user to previously acquire a sufficient amount of sterilant.

Some or all of the above embodiments may be selectively combined and configured so that various modifications are possible.

Further, the above embodiments are merely intended to describe the present invention and are not intended to limit the invention. Furthermore, those skilled in the art will appreciate that various embodiments are possible within the M scope of the technical spirit of the present invention.

What is claimed is:

1. A system for remotely controlling and managing sterilizer, comprising:
N sterilizers configured to sterilize sterilization targets;
a mobile terminal configured to receive a control command for each sterilizer or display information related to the sterilizer; and
an integrated sterilizer management server connected to the N sterilizers over a network, and configured to transmit the control command received from the mobile terminal to the corresponding sterilizer, allow the corresponding sterilizer to be driven in compliance with the control command, collect current status information of the N sterilizers, and transmit the collected current status information to the mobile terminal.

2. The system of claim 1, wherein the integrated sterilizer management server comprises:
a management server for processing and managing commands related to the sterilizers; and
middleware for relaying information stored in or managed by the management server.

3. The system of claim 2, wherein the management server comprises:
a sterilizer communication data transmission/reception module for transmitting/receiving data to/from the N sterilizers in real time;
a sterilizer type classification module for analyzing data received from the N sterilizers, classifying types of the sterilizers and registration or non-registration of the sterilizers, and converting data to be transmitted to a corresponding sterilizer into a data format corresponding to a type of sterilizer if there is data to be transmitted to the sterilizer;
a monitoring data processing module for analyzing the data received from the N sterilizers, acquiring information about at least one of processing status, Input/Output (I/O) status, power status, alarm status, and cycle information of the corresponding sterilizer, and storing the acquired information in a given database (DB);
a control command data processing module for classifying control commands for the sterilizers, determining whether the sterilizers are controllable, processing transmitted/received control commands, and storing results of control based on the transmitted/received control commands in the DB;
a user interface module for setting sterilizers or users, and displaying cycles, alarm or control status;
a sterilization cycle management module for, when a sterilization cycle-related request command is input, searching the DB for respective periods/sterilizer types/sterilizer numbers in relation to a sterilization cycle, and extracting found data based on the search;
an alarm management module for, when a current sterilizer alarm condition checking command is input, searching for and extracting current alarm occurrence conditions of the sterilizers for respective periods/sterilizer types/sterilizer numbers;
a setting management module for managing registration/change/deletion of sterilizers, registration/change/deletion of users, or configuration of the management server;
a control management module for, when a sterilizer control history search command is input, searching for and extracting control history and control results for respective periods/sterilizer types/sterilizer numbers; and
the DB for storing data used by the remote sterilizer control and management system.

4. The system of claim 2, wherein the middleware comprises:
a DB interface module used to access the DB of the management server;
a power control module configured to transmit a command for power control from a user to the management server or receive results of power control from the management server;
a cycle control module configured to transmit a command for cycle control from the user to the management server or receive results of cycle control from the management server;
a monitoring module configured to receive real-time operating status of a sterilizer selected by the user from the management server;
an alarm processing module configured to transmit alarm occurrence information to a registered mobile terminal when an alarm occurs in a specific sterilizer;
a user authentication module configured to process user authentication; and
a communication data processing module configured to be capable of transmitting/receiving data to/from the mobile terminal in real time and convert data to be transmitted/received in conformity with a data format of the mobile terminal.

5. The system of claim 1, wherein each of the sterilizers comprises:
a main control module including a second transmission/reception unit capable of transmitting/receiving data to/from the integrated sterilizer management server when operating power of the main control module is turned on, a monitoring device for monitoring status of the sterilizer, a main control unit for controlling an operation of the sterilizer, and a power status output unit for outputting a power control command for the main control module; and
a power control module including a first transmission/reception unit capable of transmitting/receiving data to/from the integrated sterilizer management server when the operating power of the main control module is turned off, a power supply switching unit for supplying power to the main control module or cutting off the supply of power to the main control module in compliance with a control command from a power control unit, a power status monitoring unit for monitoring a power control command for the main control module, output from the power status output unit, and the power control unit for controlling the power supply switching unit so that power is supplied to the main control module or the supply of the power to the main control module is cut off in compliance with the power control command for the main control module, received from the integrated sterilizer management server or the power status monitoring unit.

6. The system of claim 1, wherein the mobile terminal comprises a user input unit capable of receiving a command required to control an operation of a corresponding sterilizer.

7. The system of claim 1, wherein the mobile terminal is configured to, when a test cycle command for activating a cycle enabling status of each sterilizer to be inspected is remotely input before a sterilization cycle is activated, transmit the input test cycle command to the integrated sterilizer management server, receive status information of the sterilizer from the integrated sterilizer management server, and display the status information of the sterilizer.
